(19) United States
(12) Patent Application Publication
(10) Pub. No.: US 2014/0186271 A1
(43) Pub. Date: Jul. 3, 2014

(54) REDUCING DENTAL CARIES

(71) Applicant: Robert Quivey, JR., Webster, NY (US)

(72) Inventor: Robert Quivey, JR., Webster, NY (US)

(73) Assignee: UNIVERSITY OF ROCHESTER, Rochester, NY (US)

(21) Appl. No.: 14/110,211

(22) PCT Filed: Apr. 5, 2012
(86) PCT No.: PCT/US12/32383

§ 371 Date: Nov. 11, 2013

Related U.S. Application Data
(60) Provisional application No. 61/473,205, filed on Apr. 8, 2011.

(51) Int. Cl. A61K 8/49; A61Q 11/00

(52) U.S. Cl. CPC A61K 8/498; A61Q 11/00
USPC 424/49

(57) ABSTRACT

Provided are methods of reducing the occurrence of dental caries in a subject. The methods comprise selecting a subject with or at risk of developing dental caries and contacting the oral cavity of the subject with a therapeutically effective amount of an HMG-CoA reductase inhibitor. Also provided are oral treatment forms comprising a statin or derivative thereof.

### CROSS REFERENCE TO RELATED APPLICATIONS

This application claims the benefit of U.S. Provisional Application No. 61/473,205, filed on Apr. 8, 2011, which is incorporated by reference herein in its entirety.

### STATEMENT REGARDING FEDERALLY FUNDED RESEARCH

This invention was made with government support under Grant No. DE0-017425-01A1 awarded by the National Institute of Dental and Craniofacial Research. The Government has certain rights in this invention.

### BACKGROUND

Dental caries, otherwise known as tooth decay or a cavity, is a disease where bacteria damage the hard tooth structure. The hard tooth structure progressively breaks down due to processes in the bacteria producing holes in the teeth. Two types of bacteria are primarily responsible for dental caries, Streptococcus and Lactobacillus. If left untreated, the disease can lead to pain, tooth loss, and death in extreme cases. Dental caries is on the rise, globally, which has been attributed to large increases in carbohydrate consumption.

### SUMMARY

Provided are methods of reducing the occurrence of one or more dental caries in a subject. The methods comprise selecting a subject with or at risk of developing dental caries and contacting the oral cavity of the subject with a therapeutically effective amount of an HMG-CoA reductase inhibitor.

Also provided are oral treatment forms comprising a statin or derivative thereof and kits comprising the disclosed oral treatment forms and an applicator.

The details of one or more embodiments are set forth in the accompanying drawings and the description below. Other features, objects, and advantages will be apparent from the description and drawings, and from the claims.

### DESCRIPTION OF DRAWINGS

FIG. 1 shows a graph demonstrating the effects of simvastatin on the growth of Streptococcus mutans growing in planktonic cultures.

FIG. 2 shows a graph demonstrating the effects of simvastatin on biofilm formation of Streptococcus mutans strain UA159.

### DETAILED DESCRIPTION

Provided are methods of reducing the occurrence of dental caries in a subject. The methods comprise selecting a subject with or at risk of developing dental caries and contacting the oral cavity of the subject with a therapeutically effective amount of an HMG-CoA reductase inhibitor.

Dental caries, otherwise known as tooth decay or dental cavities, constitutes a disease wherein bacterial processes damage hard tooth structure, for example, enamel, dentin and/or cementum. These tissues progressively break down, producing cavities or holes in the tooth. As used herein dental caries includes one or more occurrence in a subject.

A reduction in the occurrence of dental caries in a subject does not have to be complete and can be at least about 10%, 20%, 30%, 40%, 50%, 60, 70%, 80%, 90%, 95%, 100% or any other percentage reduction in between these percentages as compared to a control subject.

HMG-CoA reductase or hydroxymethylglutaryl-CoA reductase is an enzyme expressed by bacteria such as Streptococcus mutans. The HMG-CoA reductase of Streptococcus mutans strain UA159 is provided herein as an example of an enzyme that is involved in producing the building blocks of polyketides in bacteria. The protein sequence is available under GenBank Accession No. NP_{—}721341.1. In the methods set forth herein, the HMG-CoA reductase inhibitor can inhibit or reduce the activity of the HMG-CoA reductase expressed by oral streptococci as compared to the activity level in the absence of the inhibitor.

Optionally, the HMG-CoA reductase inhibitor is a statin or derivative thereof. The statin or derivative thereof can be selected from the group consisting of simvastatin, atorvastatin, cerivastatin, fluvastatin, lovastatin, mevastatin, pitavastatin, pravastatin, rosuvastatin, and derivatives thereof, or a pharmaceutically acceptable salt thereof. Statins are known in the art, see, e.g., Statins: Understanding Clinical Use; Mehta, J., ed.; Elsevier, Inc.; Philadelphia, Pa. (2004). Methods of making statins are also known in the art. See, for example, U.S. Pat. No. 4,444,784 and U.S. Pat. No. 4,346,227, which are both incorporated herein in their entireties by this reference.

Pharmaceutically acceptable salts are those salts derived from pharmaceutically acceptable inorganic and organic acids and bases. These include but are not limited to, acetate, alginate, ascorbate, aspartate, benzoate, bicarbonate, bisulfate, bitartrate, horate, bromide, butyrate, calcium edetate camphorate, camphorsulfonate, camsylate, carbonate, chloride, citrate, clavulanate, dihydrochloride, edetate, estolate, esylate, ethanesulfonate, formate, fumarate, gluceptate, glucoheptonate, gluconate, glutamate, glycolylarsanilate, hemisulfate, hexanoate, hexylresorcinate, hydrabamine, hydrochloride, hydroiodide, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, tannate, tartrate, teoclate, tosylate, triethiodide, undecanoate, valerate, and the like.

Optionally, the therapeutically effective amount of the HMG-CoA reductase inhibitor reduces the number of oral streptococci in the oral cavity of the subject as compared to a control. The oral streptococci can, for example, be Streptococcus mutans.

Optionally, the therapeutically effective amount of the HMG-CoA reductase inhibitor disrupts dental plaque in the oral cavity of the subject as compared to a control.

As used herein, control refers to an untreated sample or subject. The control can be the same subject before or after the effect of the treatment with the HMG-CoA reductase inhibitor, or a different untreated subject. A control can include a known value or can be a known sample run in parallel with the experimental sample.

Optionally, the HMG-CoA reductase inhibitor is an oral treatment form. The oral treatment form can be selected from the group consisting of toothpaste, mouth rinse, gel, foam, varnish, polish, floss, gum, dental tray, dental strip, copolymer membrane, and slow release bead. Such oral treatment forms include forms for personal and professional use.

In addition to the HMG-CoA reductase inhibitor, the oral compositions described herein may contain any conventional ingredient for the particular oral vehicle. For example, liquid mouthwashes or mouth rinses may contain a solvent such as distilled or deionized water, ethanol and the like; a sweetening agent such as saccharine, aspartame and the like; and a flavoring agent such as peppermint oil, spearmint oil and the like. Toothpastes or gels may contain, for example, water, glycerine or sorbitol, a conventional abrasive such as calcium pyrophosphate, aluminum hydroxide, resins, insoluble alkali metal metaphosphates and the like in a standard amount of 20-60% wt.; a binder such as hydroxyethyl cellulose, xanthin gum, sodium carboxymethyl cellulose and the like in a standard amount of 0.5-5.0% wt.; a foaming agent such as sodium lauryl sulfate, sodium coconut monoglyceride sulfonate, sodium-N-methyl-N-palmitoyl tauride and the like in a standard amount of 0.5-3.0% wt.; a flavoring agent; a sweetening agent; an antiseptic agent and any other ingredient required for the particular formulation. A chewing gum composition can include any variety of different chewing gum types such as, for example, low and high moisture, sugar or sugarless, wax-containing or wax-free, low calorie, and the like. In the methods set forth herein, a polish can be a fluoride paste or any other polishing substance, for example, a professional fluoride polish utilized in a dentist's office. Any of the oral compositions described herein can also comprise additional ingredients useful in the promotion of dental health and hygiene, including plaque- and calculus- inhibiting, dental cleaning, whitening, or odor reducing agents, and the like.

Optionally, the HMG-CoA reductase inhibitor is in the form of an oral supplement. An oral supplement can be, but is not limited to, a liquid solution, suspension, emulsion, capsule, lozenge, sustained release formulation, or powder. The oral supplement is not a pill or a tablet. The compositions will include a therapeutically effective amount of an HMG-CoA reductase inhibitor in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The preparation of pharmaceutically acceptable carriers and formulations containing these materials is described in, e.g., Remington's Pharmaceutical Sciences, 21^{st} Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

For solid compositions (e.g., powder or capsule forms), conventional non-toxic solid carriers can include, for example, pharmaceutical grades of mannitol, lactose, starch, sodium saccharine, cellulose, magnesium carbonate, or magnesium stearate.

The HMG-CoA reductase inhibitor can, for example, be present in the oral supplement in a dosage lower than the dosage used to reduce cholesterol and triglycerides in a subject. Optionally, the HMG-CoA reductase inhibitor is present in the oral supplement in a dosage of less than 1 milligram/milliliter (mg/ml). Optionally, the HMG-CoA reductase inhibitor is present in the oral supplement in a dosage range of about 100 micrograms/ml (μg/ml) to about 500 micrograms/ml (μg/ml). Optionally, the HMG-CoA reductase inhibitor is present in the oral supplement in a dosage range of about 100 micrograms/ml (μg/ml) to about 250 micrograms/ml (μg/ml). Optionally, the HMG-CoA reductase inhibitor is present in the oral supplement in a dosage range of about 100 micrograms/ml (μg/ml) to about 150 micrograms/ml (μg/ml). Optionally, the HMG-CoA reductase inhibitor is present in the oral supplement in a dosage range of about 50 micrograms/ml (μg/ml) to about 100 micrograms/ml (μg/ml). Optionally, the HMG-CoA reductase inhibitor is present in the oral supplement in a dosage range of about 25 micrograms/ml (μg/ml) to about 50 micrograms/ml (μg/ml). Optionally, the HMG-CoA reductase inhibitor is present in the oral supplement in a dosage renage about about 10 micrograms/ml (μg/ml) to about 25 micrograms/ml (μg/ml). Optionally, the HMG-CoA reductase inhibitor is present in the oral supplement in a dosage of about 100 μg/ml, 90 μg/ml, 80 μg/ml, 70 μg/ml, 60 μg/ml, 50 μg/ml, 40 μg/ml, 30 μg/ml, 25 μg/ml, 20 μg/ml, 15 μg/ml, or about 10 μg/ml.

Also provided are oral treatment forms comprising a statin or derivative thereof. The statin or derivative thereof can, for example, be present in an amount less than or equal to about 1 mg per unit dose. The statin or derivative thereof can also be present in an amount of about 100 μg to about 500 μg, in an amount of about 100 μg to about 250 μg, or in an amount of about 100 μg to about 150 μg per unit dose. The statin or derivative thereof can also be present in an amount of about 50 μg to about 100 μg, in an amount of about 25 μg to about 75 μg, in an amount of about 25 μg to about 50 μg per unit dose, or in an amount of about 10 μg to about 25 μg per unit dose. Optionally, the statin or derivative thereof is present in an amount less than or equal to about 100 μg per unit dose. Optionally, the statin or derivative thereof is present in an amount less than or equal to about 50 μg per unit dose. Optionally, the statin or derivative thereof is present in an amount less than or equal to about 25 μg per unit dose. Optionally, the oral treatment form is selected from the group consisting of toothpaste, mouth rinse, gel, gum, foam, varnish, polish, floss, dental tray, dental strip, copolymer membrane, and slow release bead. By unit dose is meant one volume of toothpaste, mouth rise, gel, gum, foam, varnish, polish, floss, dental tray, dental strip, copolymer membrane, and slow release bead, as typically used by a subject in one use. By unit dose is meant a prescribed amount for a single prescription according to the directions for the oral prescription form or an average range for a self-administered or self-selected single treatment.

Also provided are kits comprising the oral treatments described herein and an applicator. The applicator can be, for example, a toothbrush, a dental tray, a dispenser or a spray bottle.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult and newborn subjects, whether male or female, are intended to be covered. As used herein, patient or subject may be used interchangeably and can refer to a subject with a disease or disorder or at risk of a disease or disorder (e.g., dental caries). The term patient or subject includes human and veterinary subjects.

One of skill in the art can identify a subject at risk of developing dental caries. For example, a subject at risk of developing dental caries can be predisposed to the dental caries (e.g., have a personal or family history or have a mutation in a gene that reduces enamel or otherwise causes dental caries) or show early signs or symptoms of dental caries. A subject at risk of developing dental caries can also be at risk due to environmental factors (e.g., poor oral hygiene, a diet high in sugar, other factors that reduce enamel, limited access to dental care, or absence of fluoridated water). Visual inspection, fluorescence imaging, X-rays, and/or medical/dental history can reveal a subject with or at risk of dental caries. A caries activity test (Cariostat) can also be employed. The surface of the tooth may be soft when probed with a sharp instrument. One of skill in the art can also obtain a saliva or dental plaque sample from the subject and determine the presence or absence of a particular bacteria, for example Streptococcus mutans, in order to determine the risk of developing dental caries. Also, a subject currently with dental caries has one or more than one symptom and may have been diagnosed with the dental caries.

The methods and agents as described herein are useful for both prophylactic and therapeutic treatment. For prophylactic use, a therapeutically effective amount of the agents described herein are administered to a subject prior to onset (e.g., before obvious signs of dental caries) or during early onset (e.g., upon initial signs and symptoms of dental caries). Prophylactic administration can occur for several days to years prior to the manifestation of symptoms of dental caries. Prophylactic administration can be used, for example, in the preventative treatment of subjects at risk for dental caries. Therapeutic treatment involves administering to a subject a therapeutically effective amount of the agents described herein after diagnosis or development of one or more dental caries.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the following claims.

### EXAMPLES

### Materials and Methods

Bacterial Strain. The strain of Streptococcus mutans used in these experiments is referred to as UA159, the genomic type strain of these organisms (Murchison et al., Infect. Immun. 54(2):273-82 (1986); Ajdic et al., Proc. Natl. Acad. Sci. USA 99(22):14434-9 (2002)).
Growth experiments. Two types of growth experiments were conducted: growth of the organism in planktonic culture and growth of the organism in a mono-species biofilm culture. In the planktonic culture experiments, the effects of simvastatin concentration on the growth ability of Streptococcus mutans UA159 was determined. In the second set of experiments, the effects of simvastatin concentration on the ability of S. mutans to produce extracellular polysaccharides (EPS), which is highly related to its ability to grow and survive on the surfaces of teeth (Yamashita et al., Infect. Immun 61(9):3811-7 (1993)), was determined.

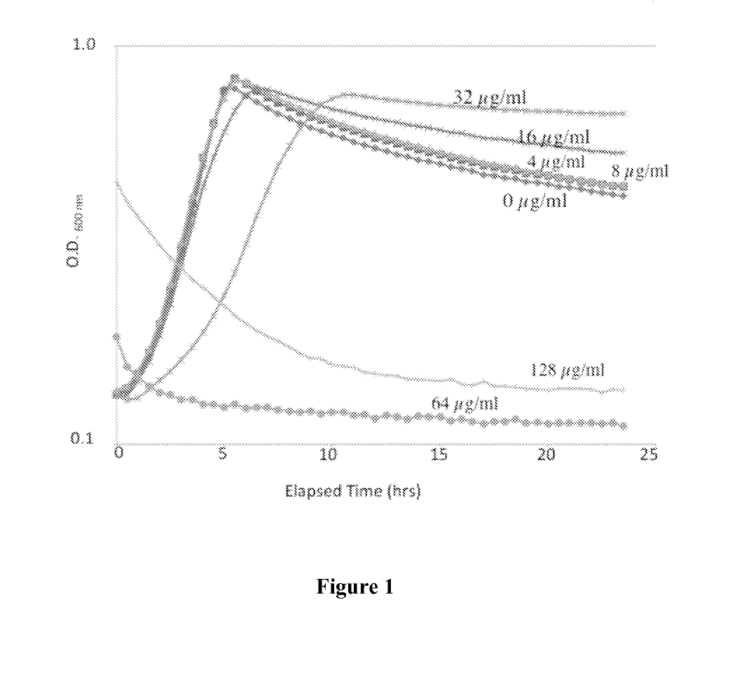

The planktonic experiments were conducted by growing S. mutans in Brain Heart Infusion medium (BHI) supplemented with specific concentrations of simvastatin dissolved in 1% ethanol/99% water. Simvastatin was obtained from Sigma Chemical Co. (St. Louis, Mo.). Growth was monitored as increasing optical density at 600 nm, using a BioScreen C automated-recording instrument, set at 37° C. and with shaking set at 10 seconds, every 15 minutes, to ensure suspension of the culture (Growth Curves USA, N.J.). The resulting turbidometric data were plotted against time, as shown in FIG. 1.

Biofilm cultures were prepared in 96-well microtitre-plates, 200 microliter (μl) volumes, using medium composed of tryptone yeast extract (TY) supplemented with either 1% (w/v) glucose, TYG, or with 1% (w/v) sucrose, TYS. The cultures were also supplemented with simvastatin at specific concentrations prepared in 1% ethanol, balance sterile double-distilled water. The culture wells were inoculated using an overnight culture of S. mutans UA159 as the source of cells. The plates were then placed in a 37° C. incubator with a carbon-dioxide enriched atmosphere (5% CO₂/95% air) and incubated for 24 hours. Following growth, spent medium and cells were removed by rinsing the wells with sterile water. The production of biofilm was estimated using the crystal violet-dye binding assay as described previously (Loo et al., J. Bacteriol. 182(5):1374-82 (2000)).

### EXAMPLE 1

### The HMG-CoA Reductase Inhibitor, Simvastatin, Affects the In Vitro Growth of Streptococcus mutans

HMG-CoA reductase mutational studies performed on Streptococcus mutans, revealed that the protein subunits of HMG-CoA reductase were important for the growth of the organism. Mutation of mvaA rendered S. mutans highly sensitive to growth in acidic conditions, similar to those found in a disease state, whereas mutation of mvaS was a lethal event for S. mutans.

To determine whether HMG-CoA reductase inhibitors had an effect on S. mutans growth, increasing amounts of simvastatin were administered to planktonic cultures of S. mutans. The results from the measurements of S. mutans growth in the presence of various concentrations of simvastatin are shown in FIG. 1. The presence of the carrier, 1% ethanol, did not affect growth of the organism under these conditions.

Increasing concentrations of simvastatin reduced the growth of S. mutans strain UA159 growing in rich medium in planktonic culture as evidenced by FIG. 1. The higher optical density of the 128 μg/ml line is likely due to the formation of micelles by simvastatin (soap bubbles). Growth was effectively halted at 64 μg/ml and above. Importantly, simvastatin did not kill the bacteria, and it was observed during microbiological plating of the bacteria that the survival of the bacterium was essentially identical in cultures incubated with 0 or 128 μg/ml, which confirms that simvastatin, like fluoride, is bacteriostatic.

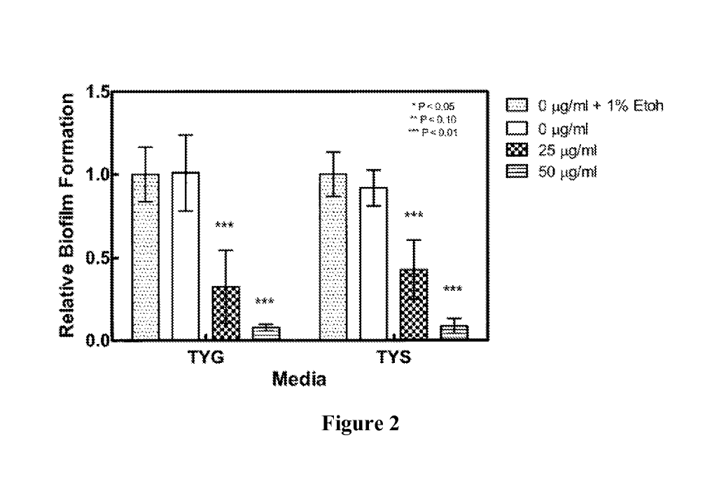

To determine whether HMG-CoA reductase inhibitors were capable of affecting S. mutans growth in the more natural habitat of the organism, increasing amounts of simvastatin were administered to biofilms of S. mutans strain UA159. The EPS measurements, using S. mutans grown in the presence of sucrose or glucose, showed dramatic differences when simvastatin was included in the growth medium. FIG. 2 shows increased values for production of polysaccharide when the S. mutans was grown in the presence of sucrose and the absence of simvastatin. Sucrose is the preferred substrate of the enzymes produced by S. mutans. Those enzymes, referred to as glucosyltransferases, utilize the glucose moiety of sucrose to form a complex glucan polymer, which in turn forms the carbohydrate matrix dental plaque. In the presence of simvastatin, the production of EPS is reduced (FIG. 2). Based on the results of the experiments depicted in FIG. 1, it would suggest that the bacteria in the EPS experiments did not grow in the presence of simvastatin, and hence were unable to produce the extracellular enzymes that generate the EPS. The data of FIG. 2 indicate that EPS production was reduced in the presence of simvastatin, suggesting that the pathogenic capability of the organism under these conditions would also be reduced.

Using well established methodologies, the minimum inhibitory concentration of simvastatin was determined against a panel of human pathogens. The data demonstrated that S. mutans was more sensitive to simvastatin compared to the other bacterial species (Table 1).

TABLE 1
Minimal inhibitory concentration (MIC) of simvastatin for S. mutans is
lower than that for other human pathogens

| Pathogen | MIC (μg/ml) |
|---|---|
| S. aureus | 64 μg/ml |
| Klebsiella | 64 μg/ml |
| E. faecium | 64 μg/ml |
| E. faecalis | 64 μg/ml |
| P. aeruginosa | >256 μg/ml |
| S. mutans | 16 μg/ml |

### EXAMPLE 2

### The HMG-CoA Reductase Inhibitor, Simvastatin, Affects the In Vivo Growth of Streptococcus mutans

Utilizing a well-established rat model of dental caries described previously (Fozo et al., Infect. Immun. 75(3):1537-9 (2007)), groups of 15 rat pups are orally infected with S. mutans UA159. All animals are infected and provided a high sucrose diet (Diet-2000) and water ad libitum. One group of animals is not treated further; an additional group is treated by bushing the teeth twice daily with a solution containing 50 μg/ml simvastatin, and a third group is treated by brushing the teeth twice daily with a solution containing 1% alcohol/99% water as a carrier control. At the end of five weeks, the animals are sacrificed and established methods for scoring enamel and sulcul caries are performed.

1. A method of reducing the occurrence of dental caries in a subject comprising
(a) selecting a subject with or at risk of developing dental caries and
(b) contacting the oral cavity of the subject with a therapeutically effective amount of an HMG-CoA reductase inhibitor.

2. The method of claim 1, wherein the HMG-CoA inhibitor reductase is a statin or a derivative thereof.

3. The method of claim 2, wherein the statin or derivative thereof is selected from the group consisting of simvastatin, atorvastatin, cerivastatin, fluvastatin, lovastatin, mevastatin, pitavastatin, pravastatin, rosuvastatin, and derivatives thereof.

4. The method of claim 2, wherein the statin is simvastatin or a derivative thereof.

5. The method of claim 1, wherein the therapeutically effective amount of the HMG-CoA reductase inhibitor reduces the number of oral streptococci in the oral cavity of the subject as compared to a control.

6. The method of claim 5, wherein the oral streptococci is Streptococcus mutans.

7. The method of claim 1, wherein the therapeutically effective amount of the HMG-CoA reductase inhibitor disrupts dental plaque in the oral cavity of the subject.

8. The method of claim 1, wherein the HMG-CoA reductase inhibitor is an oral treatment form selected from the group consisting of a toothpaste, mouth rinse, gel, foam, varnish, polish, floss, dental tray, dental strip, copolymer membrane, and slow release bead.

9. The method of claim 1, wherein the HMG-CoA reductase inhibitor is in the form of an oral supplement.

10. The method of claim 9, wherein the HMG-CoA reductase inhibitor is present in the oral supplement in a dosage lower than the dosage used to reduce cholesterol and triglycerides.

11. The method of claim 10, wherein the HMG-CoA reductase inhibitor is present in the oral supplement in a dosage of less than 1 mg/ml.

12. The method of claim 10, wherein the HMG-CoA reductase inhibitor is present in the oral supplement in a dosage of less than 100 μg/ml.

13. The method of claim 10, wherein the HMG-CoA reductase inhibitor is present in the oral supplement in a dosage of 50 μg/ml.

14. The method of claim 10, wherein the HMG-CoA reductase inhibitor is present in the oral supplement in a dosage of 25 μg/ml.

15. An oral treatment form comprising a statin or derivative thereof.

16. The oral treatment form of claim 15, wherein the oral treatment form is selected from the group consisting of a toothpaste, mouth rinse, gel, foam, varnish, polish, floss, dental strip, copolymer membrane, and slow release bead.

17. The oral treatment form of claim 15, wherein the statin is present in an amount of less than 1 mg.

18. The oral treatment form of claim 15, wherein the statin is present in an amount of less than 100 μg.

19. The oral treatment form of claim 15, wherein the statin is present in an amount of 50 μg.

20. The oral treatment form of claim 15, wherein the statin is present in an amount of 25 μg.

21. A kit comprising the oral treatment form of claim 15 and an applicator.

22. The kit of claim 21, wherein the applicator is a toothbrush or dental tray.
